(19) United States
(12) Patent Application Publication
(10) Pub. No.: US 2025/0170352 A1
(43) Pub. Date: May 29, 2025

(54) TWO-COLOR MOLDED ANTI-SLIP GRIP RESPIRATORY MASK AND MOLD THEREFOR

(71) Applicant: HSINER CO., LTD., Taichung City (TW)

(72) Inventor: Eric CHANG, Taichung City (TW)

(21) Appl. No.: 18/434,584

(22) Filed: Feb. 6, 2024

(30) Foreign Application Priority Data
Nov. 24, 2023 (TW) 112145607

(51) Int. Cl. A61M 16/06; B29C 45/13; B29C 45/16; B29C 45/26; B29L 31/00

(52) U.S. Cl. CPC A61M 16/0611; B29C 45/13; B29C 45/1676; B29C 45/2602; B29L 2031/753

(57) ABSTRACT

A respiratory mask includes a mask body injection molded from a first composition, and a buffer member injection molded from a second composition and connected to and surrounding the mask body. The mask body includes a shield portion having an air inlet, a flange extending outwardly from the shield portion, and a grip portion cooperating with the flange and the shield portion to define a receiving groove. The shield portion further defines a facial space communicating with the air inlet. The flange has an inner surface proximate to the facial space, and an outer surface opposite to the inner surface. The grip portion protrudes from the outer surface of the flange. A mold for making the two-color molded anti-slip grip respiratory mask is also disclosed.

### CROSS-REFERENCE TO RELATED APPLICATION

This application claims priority to Taiwanese Invention Patent Application No. 112145607, filed on Nov. 24, 2023, and incorporated by reference herein in its entirety.

### FIELD

The present disclosure relates to a respiratory mask, and more particularly to a two-color molded anti-slip grip respiratory mask and a mold therefor.

### BACKGROUND

A three-plate mold, as disclosed in Taiwanese Utility Model Patent No. M428843U, includes an upper fixing plate, a stripper plate, and a female mold plate arranged in sequence from top to bottom. The upper fixing plate is provided with a pouring channel. The stripper plate is provided with a first passage for communicating with the pouring channel. The female mold plate is provided with a second passage for communicating with the first passage, and a female mold cavity communicating with the second passage. To use the three-plate mold, the upper fixing plate, the stripper plate, and the female mold plate are first pressed tightly against each other, after which molten injection molding raw material is injected from the pouring channel into the female mold cavity through the second passage to form a product in the female mold cavity. Next, the upper fixing plate, the stripper plate, and the female mold plate are separated from each other, and the residue formed in the second passage is pushed away from the stripper plate by a biasing piece. The three-plate mold has the disadvantages of high mold cost, large volume, high raw material costs, and producing large amount of residue after injection molding. Moreover, since the structure of the three-plate mold is complicated, and the mold size thereof is large, the structural strength of the three-plate mold is low, and the weight thereof is heavy.

### SUMMARY

Therefore, an object of the present disclosure is to provide a two-color molded anti-slip grip respiratory mask that can alleviate at least one of the drawbacks of the prior art.

According to one aspect of this disclosure, the respiratory mask includes a mask body injection molded from a first composition and a buffer member injection molded from a second composition. The mask body includes a shield portion surrounding a central axis and having an air inlet, a flange extending outwardly from an outer periphery of the shield portion, and a grip portion disposed on the flange and cooperating with the flange and the shield portion to define a receiving groove. The shield portion defines a facial space communicating with the air inlet. The flange has an inner surface proximate to the facial space, and an outer surface opposite to the inner surface. The grip portion protrudes from the outer surface of the flange in a direction parallel to the central axis. The buffer member is substantially ring-shaped, is connected to and surrounds the mask body, and has a hardness smaller than that of the mask body.

Another object of the present disclosure is to provide a mold for making the two-color molded anti-slip grip respiratory mask that can alleviate at least one of the drawbacks of the prior art.

According to another aspect of this disclosure, the mold is provided for making at least one two-color molded anti-slip grip respiratory mask which includes a mask body configured to be injection molded from a first composition and a buffer member configured to be injection molded from a second composition. The mask body includes a shield portion, a flange connected to the shield portion, and a grip portion protruding from the flange. The buffer member is connected to and surrounds the mask body, and has a hardness smaller than that of the mask body. The mold includes male and female molds. The male mold includes a male seat body having a first inner wall, and at least one first male mold unit disposed on the male seat body and having a first mold cavity extending inwardly from the first inner wall and defined by a first male mold surface, and a first male mold hole formed in the first inner wall and adjacent to and communicating with the first mold cavity.

The female mold includes a female seat body having a second inner wall, a first channel unit formed in the female seat body for injection of the first composition, a second channel unit formed in the female seat body for injection of the second composition and spaced apart from the first channel unit, and at least one first female mold unit and at least one second female mold unit spacedly disposed on the female seat body. The at least one first female mold unit has a first female mold surface projecting from the second inner wall. The at least one second female mold unit has a second female mold surface projecting from the second inner wall, and a female mold hole formed in the second inner wall adjacent to the second female mold surface and communicating with the second channel unit.

When the male mold is moved toward the female mold to place the mold in a first closed position, the first male and female mold surfaces cooperatively define a first mold chamber communicating with the first male mold hole and the first channel unit for forming the mask body. The first mold chamber includes a main body region for forming the shield portion, and an extended region extended from the main body region toward the first male mold surface for forming the grip portion. The extended region is adjacent to and communicates with the first male mold hole.

When the male mold is moved toward the female mold to place the mold in a second closed position, the first male and female mold surfaces cooperatively define a product chamber and a buffer chamber communicating with the product chamber and the female mold hole for forming the buffer member.

### BRIEF DESCRIPTION OF THE DRAWINGS

Other features and advantages of the disclosure will become apparent in the following detailed description of the embodiments with reference to the accompanying drawings. It is noted that various features may not be drawn to scale.

FIG. 1 is a perspective view of a two-color molded anti-slip grip respiratory mask according to the first embodiment of the present disclosure.

FIG. 2 is a top view of the first embodiment.

FIG. 3 is a partly sectional view taken along line III-III of FIG. 2.

FIG. 4 illustrates how the first embodiment is used on a patient.

FIG. 5 is a perspective view of a two-color molded anti-slip grip respiratory mask according to the second embodiment of this disclosure.

FIG. 6 is a perspective view of a two-color molded anti-slip grip respiratory mask according to the third embodiment of this disclosure.

FIG. 7 is a perspective view of a two-color molded anti-slip grip respiratory mask according to the fourth embodiment of this disclosure.

FIG. 8 is a perspective view of a two-color molded anti-slip grip respiratory mask according to the fifth embodiment of this disclosure.

FIG. 9 is a perspective view of a mold for making the two-color molded anti-slip grip respiratory mask of this disclosure.

FIG. 10 is a fragmentary sectional perspective view of the mold of this disclosure.

FIG. 11 is an exploded perspective view of a male mold and a female mold of the mold of this disclosure.

FIG. 12 is a fragmentary front view of the male mold of this disclosure.

FIG. 13 is a fragmentary front view of the female mold of this disclosure.

FIG. 14 is a fragmentary enlarged sectional view, illustrating the mold of this disclosure in a first closed position.

FIG. 15 is a sectional view taken along line XV-XV of FIG. 14.

FIG. 16 is a fragmentary enlarged sectional view, illustrating the mold of this disclosure in a second closed position.

FIG. 17 is a sectional view taken along line XVII-XVII of FIG. 16.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

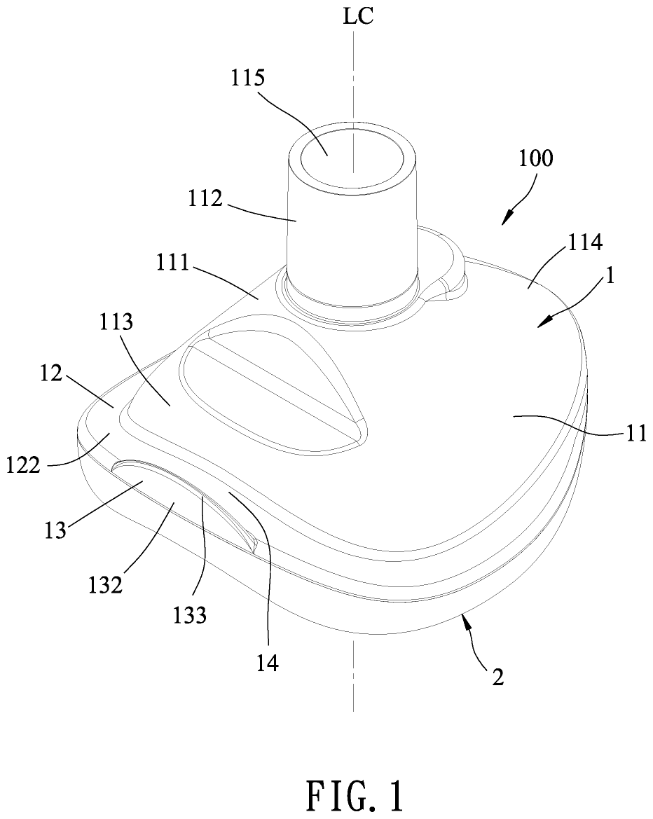

Referring to FIGS. 1 to 3, a two-color molded anti-slip grip respiratory mask 100 according to the first embodiment of the present disclosure includes a mask body 1 and a buffer member 2.

The mask body 1 includes a shield portion 11, a flange 12, and a grip portion 13. The shield portion 11 includes an intermediate section 111, an air tube 112, a jaw section 113, and a nose bridge section 114. The air tube 112 is connected to the intermediate section 111, defines a central axis (LC), and has an air inlet 115. The jaw section 113 is located at one side of the intermediate section 111. The nose bridge section 114 is located at another side of the intermediate section 111, and is opposite to the jaw section 113 along a transverse direction (D) transverse to the central axis (LC). The jaw section 113 extends in a direction transverse to the transverse direction (D). The shield portion 11 defines a facial space 116 communicating with the air inlet 115.

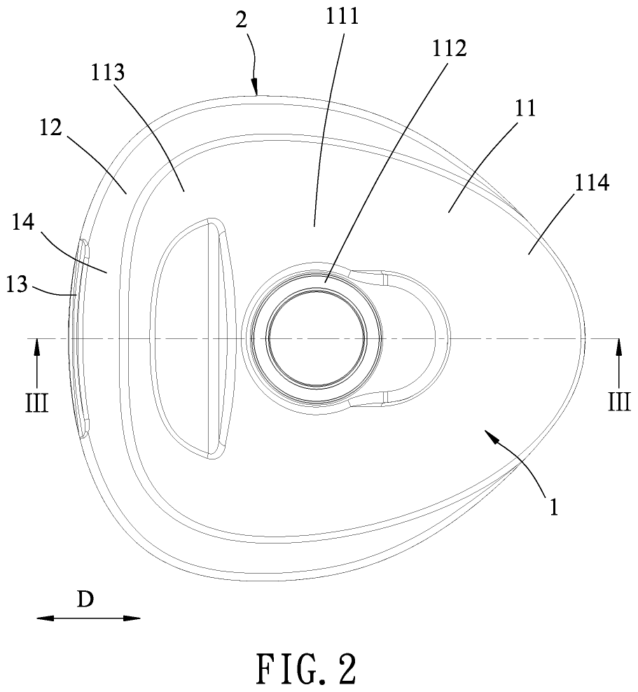

With reference to FIG. 2, the flange 12 is U-shaped that extends outwardly from and that surrounds an outer periphery of the shield portion 11. That is, the flange 12 extends along the outer periphery of the shield portion 11 from one side of the intermediate section 111 through the jaw section 113 to the other side of the intermediate section 111 that is opposite to the one side along the direction transverse to the transverse direction (D). The flange 12 has an inner surface 121 proximate to the facial space 116, and an outer surface 122 opposite to the inner surface 121.

The grip portion 13 protrudes from the outer surface 122 of the flange 12 in a direction parallel to the central axis (LC), and has an elongated plate shape that extends in the direction transverse to the transverse direction (D). The grip portion 13 has a first side surface 131 facing the jaw section 113, a second side surface 132 opposite the first side surface 131, and a connecting surface 133 connected between ends of the first and second side surfaces 131, 132 that are distal from the flange 12. The grip portion 13 cooperates with the flange 12 and the jaw section 113 to define a receiving groove 14. The first side surface 131 is adjacent to the receiving groove 14.

The buffer member 2 is substantially ring-shaped that surrounds an outer periphery of the mask body 1.

In this embodiment, the mask body 1 is injection molded from a first composition. The first composition is a polypropylene (PP) composition that becomes molten when heated. That is, the mask body 1 is a hard plastic made of polypropylene. The buffer member 2 is injection molded from a second composition. The second composition is a thermoplastic rubber (TPR) composition that becomes molten when heated. That is, the buffer member 2 is made of an elastic thermoplastic rubber, and has a hardness smaller than that of the mask body 1. Furthermore, the color of the buffer member 2 is different from that of the mask body 1.

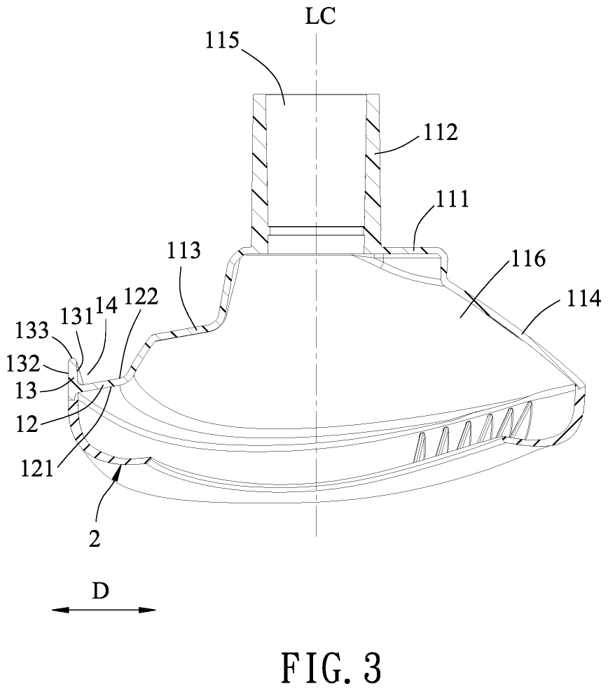

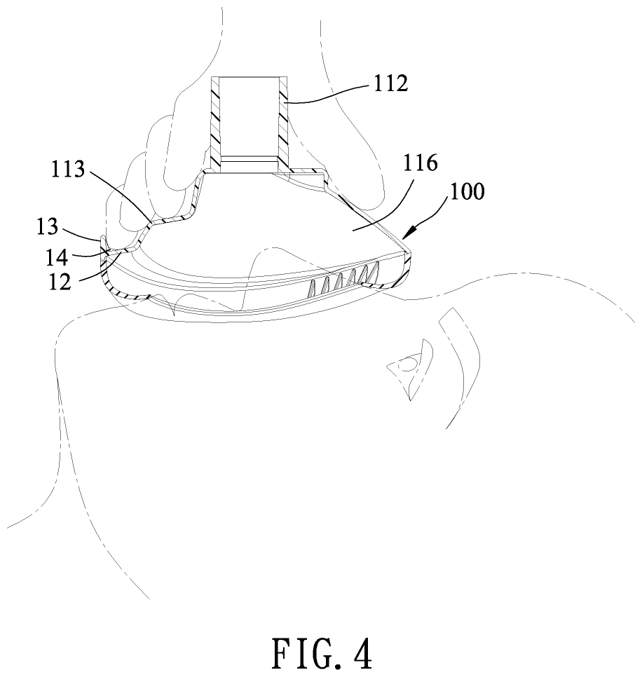

Referring to FIG. 4, in combination with FIG. 3, when a medical personnel wears the gloves and covers the respiratory mask 100 connected with an anesthesia tube (not shown) on the face of a lying patient, the mouth and nose of the patient are received in the facial space 116, and the little finger of the medical personnel is fitted into the receiving groove 14 with an outer side thereof abutting against the first side surface 131 and blocked by the grip portion 13 so as to be positioned thereat, thereby preventing the hand of the medical personnel from slipping away from the respiratory mask 100 in a direction away from the air tube 112 when adjusting the respiratory mask 100. It should be noted that, when the medical personnel holds the respiratory mask 100 with a different gesture, other fingers or palm of the medical personnel may also be retained in the receiving groove 14 to achieve the effect of avoiding slippage. Moreover, since the buffer member 2 is not an air bladder made of polyvinyl chloride, it will not have the disadvantages of the air bladder, such as the material being easily broken or easily expand or contract due to external pressure to thereby reduce the fit with the face of the patient.

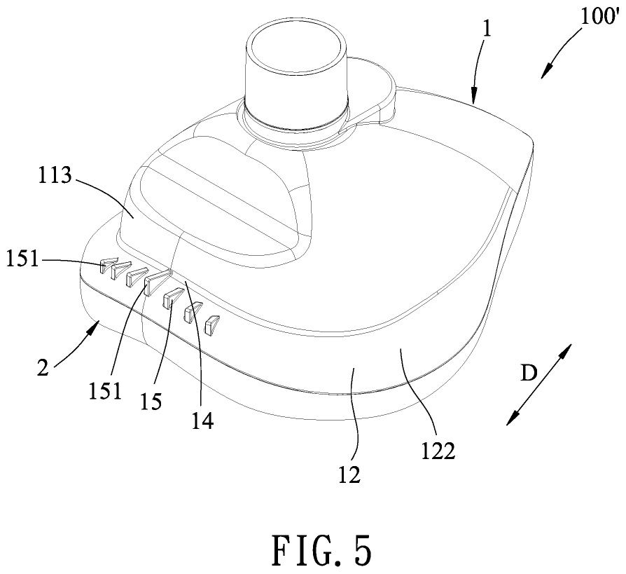

Referring to FIG. 5, the respiratory mask 100′ according to the second embodiment of this disclosure differs from the first embodiment in that the grip portion 15 of this embodiment has a plurality of protrusions 151 protruding from the outer surface 122 of the flange 12 and spacedly arranged in a direction transverse to the transverse direction (D). The finger of the medical personnel can similarly be fitted into the receiving groove 14 between the protrusions 151 and the jaw section 113 when the medical personnel covers the respiratory mask 100′ on the face of the patient. The protrusions 151 can provide support to or leaning thereagainst of the finger or palm of the medical personnel. The second embodiment can likewise achieve the same effect as the first embodiment.

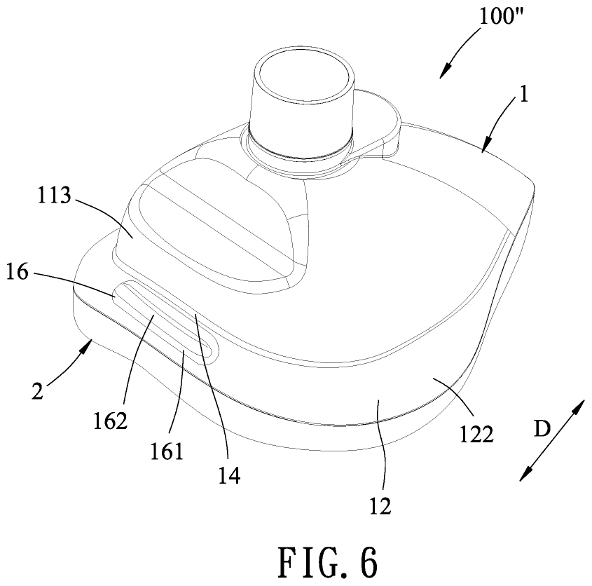

Referring to FIG. 6, the respiratory mask 100″ according to the third embodiment of this disclosure differs from the first embodiment in that the grip portion 16 of this embodiment has an elongated section 161 connected to the outer surface 122 of the flange 12 and extending in the direction transverse to the transverse direction (D), and a groove 162 recessed in and extending along a length of the elongated section 161. The finger or palm of the medical personnel can similarly be fitted into the receiving groove 14 between the grip portion 16 and the jaw section 113, and can also be fitted into the groove 162 in this case. The third embodiment can likewise achieve the same effect as the first embodiment.

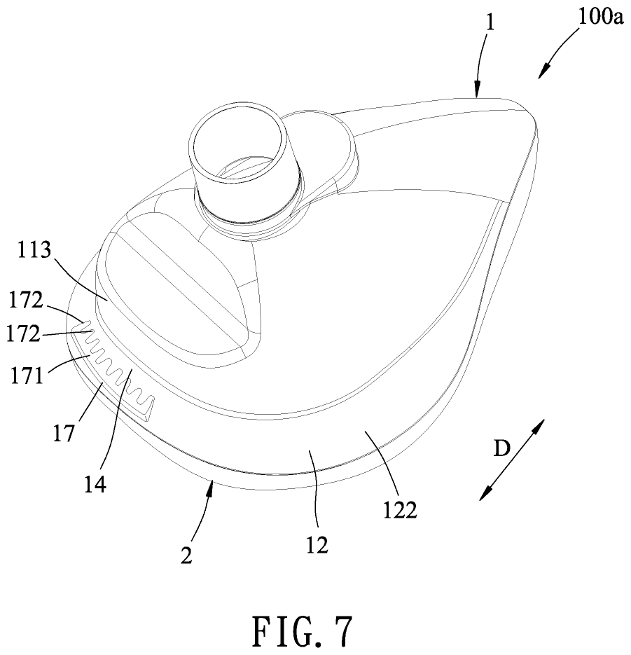

Referring to FIG. 7, the respiratory mask (100a) according to the fourth embodiment of this disclosure differs from the first embodiment in that the grip portion 17 of this embodiment has an elongated section 171 connected to the outer surface 122 of the flange 12 and extending in the direction transverse to the transverse direction (D), and a plurality of protruding sections 172 extending from the elongated section 171 toward the jaw section 113 and spaced apart from each other along a length of the elongated section 171. The finger of the medical personnel can similarly be fitted into the receiving groove 14 between the grip portion 17 and the jaw section 113. The elongated section 171 and the protruding sections 172 can provide support to or leaning thereagainst of the finger or palm of the medical personnel, and the protruding sections 172 can prevent the finger of the medical personnel from sliding along the length of the elongated section 171. The fourth embodiment can likewise achieve the same effect as the first embodiment.

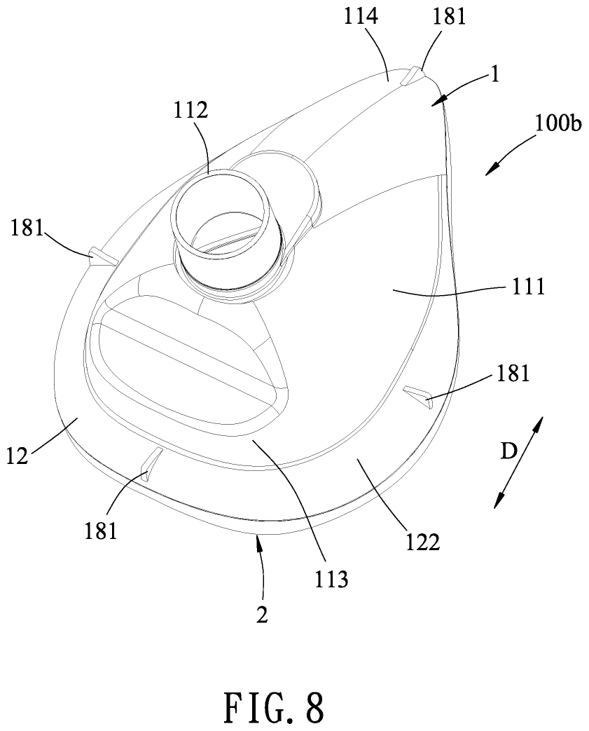

Referring to FIG. 8, the respiratory mask (100b) according to the fifth embodiment of this disclosure differs from the first embodiment in that the grip portion 18 of this embodiment has four protrusions 181, one of which is located on the outer surface 122 of the flange 12 adjacent to the jaw section 113, a second one of which is located on the nose bridge section 114, and the remaining two of which are located on the outer surface 122 of the flange 12 and are respectively adjacent to two sides of the intermediate section 111 that are opposite along the direction transverse to the transverse direction (D). The protrusions 181 can similarly provide support to or leaning thereagainst of the finger or palm of the medical personnel, and can provide abutment effect relative to four directions of the air tube 112. The fifth embodiment can likewise achieve the same effect as the first embodiment.

In summary, through the grip portion 13, 15, 16, 17, 18, the flange 12 and the jaw section 113 of the mask body 1 of the respiratory mask 100, 100′, 100″, 100a, 100b of this disclosure that cooperatively define the receiving groove 14, when the medical personnel wears the gloves and covers the respiratory mask 100, 100′, 100″, 100a, 100b on the face of the patient with different gestures or angles, the grip portion 13, 15, 16, 17, 18 can provide support to or leaning thereagainst of the hand of the medical personnel, while the receiving groove 14 can receive the finger or the palm of the medical personnel so as to prevent the hand of the medical personnel from slipping off the respiratory mask 100, 100′, 100″, 100a, 100b. As such, not only the efficiency of using the respiratory mask 100, 100′, 100″, 100a, 100b can be increased, the discomfort caused by the medical personnel applying too much pressure when putting the respiratory mask 100, 100′, 100″, 100a, 100b on the face of the patient can also be reduced. Therefore, the object of the present disclosure can indeed be achieved.

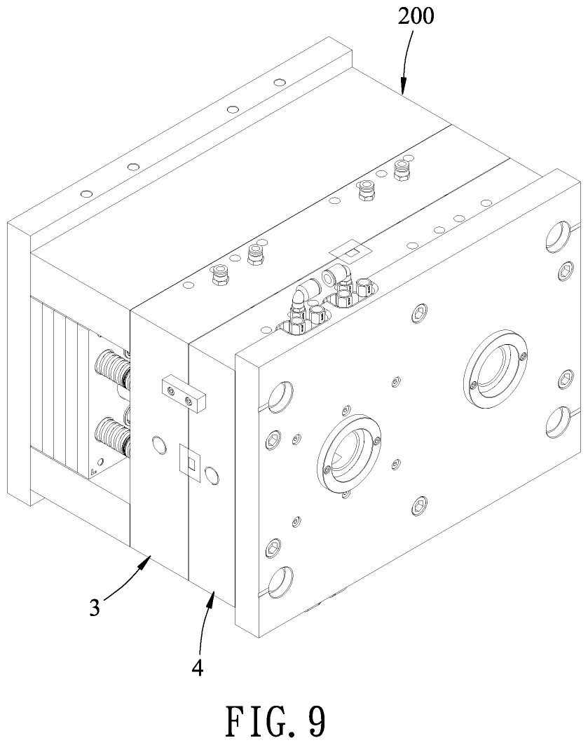

Referring to FIGS. 9 to 11, a mold 200 for making the respiratory mask 100 of the first embodiment includes a female mold 4, and a male mold 3 movable toward and away from the female mold 4 along a first axis (Y).

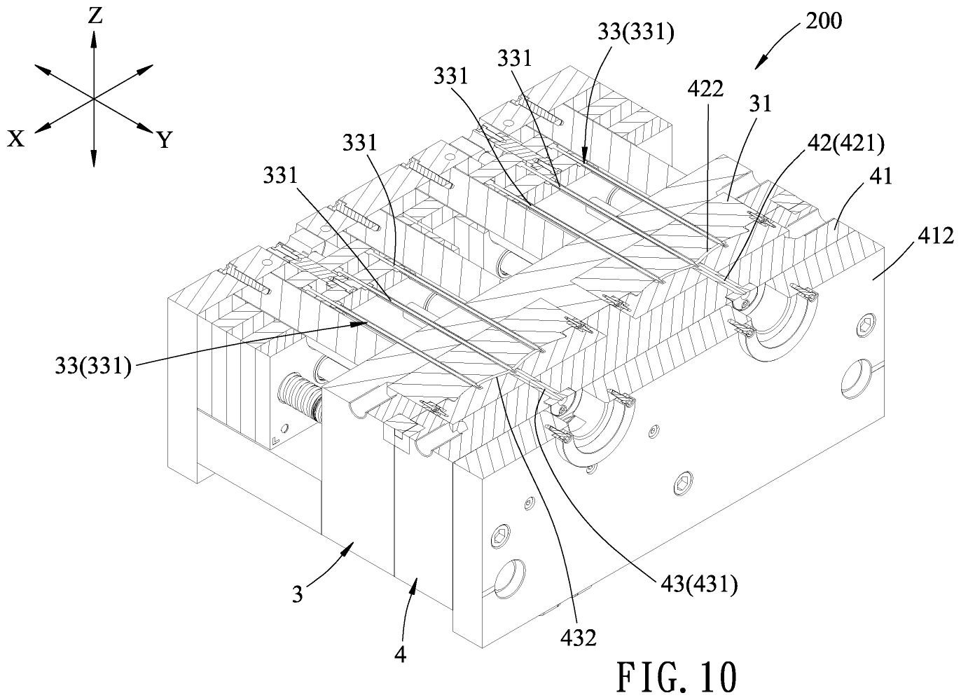

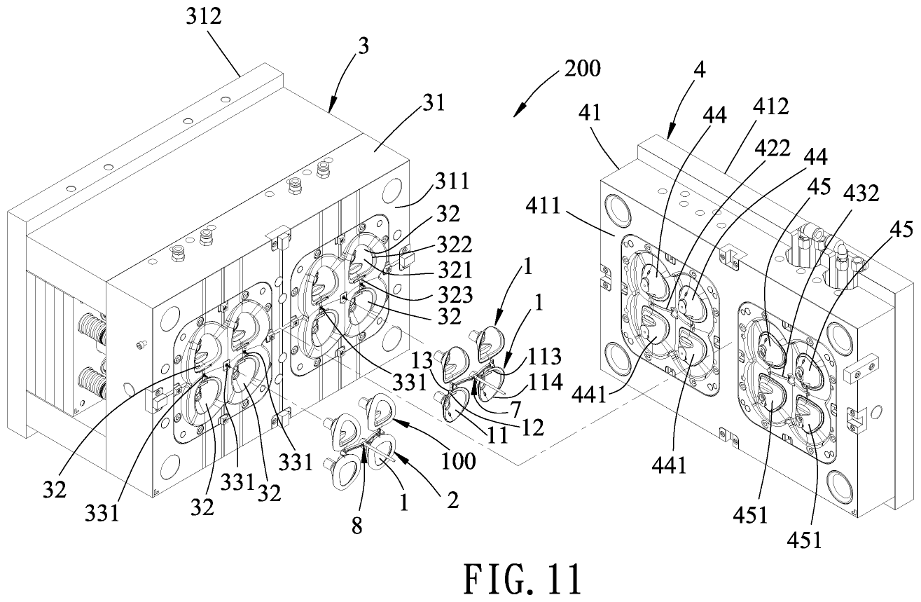

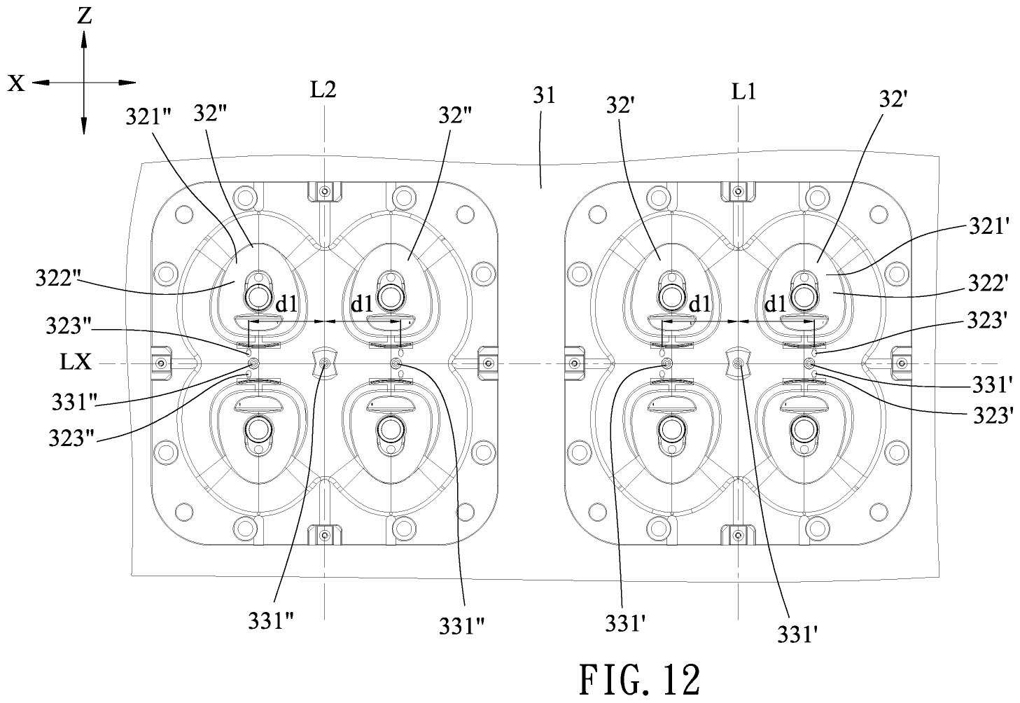

Referring to FIG. 12, in combination with FIGS. 10 and 11, the male mold 3 includes a male seat body 31 having a first inner wall 311 and a first outer wall 312 opposite to each other along the first axis (Y), eight male mold units 32 disposed on the first inner wall 311, and two ejection assemblies 33 disposed in the male seat body 31 and spaced apart from each other along a second axis (X) perpendicular to the first axis (Y). Each male mold unit 32 has a mold cavity 321 formed on the first inner wall 311 and defined by a male mold surface 322, and a male mold hole 323 formed in the first inner wall 311 and adjacent to and communicating with the mold cavity 321. Each ejection assembly 33 includes three parallel ejection rods 331 extending along the first axis (Y) and movably extending through the first inner wall 311.

To facilitate the subsequent description of the process of cooperation of the male mold 3 and the female mold 4 to form the respiratory mask 100, with reference to FIG. 12, four of the male mold units 32 disposed on one side of the first inner wall 311 are respectively defined as first male mold units 32′, while the other four male mold units 32 disposed on the other side of the first inner wall 311 are respectively defined as second male mold units 32″. Hence, the mold cavity 321, the male mold surface 322, and the male mold hole 323 of each first male mold unit 32′ are respectively defined as a first mold cavity 321′, a first male mold surface 322′, and a first male mold hole 323′; and the mold cavity 321, the male mold surface 322, and the male mold hole 323 of each second male mold unit 32″ are respectively defined as a second mold cavity 321″, a second male mold surface 322″, and a second male mold hole 323″. Furthermore, one of the ejection assemblies 33 is defined as a first ejection assembly 33′, while the other ejection assembly 33 is defined as a second ejection assembly 33″. The ejection rods 331 of the first ejection assembly 33′ are defined as first ejection rods 331′, and the ejection rods 331 of the second ejection assembly 33″ are defined as second ejection rods 331″.

The first male mold units 32′ are spaced apart from the second male mold units 32″ along the second axis (X), and are separated by a first separation axis (LX) extending parallel to the second axis (X) and a second separation axis (L1) perpendicular to the first separation axis (LX) to form a 2×2 arrangement. The second separation axis (L1) extends parallel to a third axis (Z) perpendicular to the first and second axes (Y, X). The first male mold units 32′ located on two sides of the first separation axis (LX) are symmetrical mirror images of each other. The first male mold hole 323′ of each first male mold unit 32′ is located between the first separation axis (LX) and the first male mold surface 322′ of a respective first male mold unit 32′, and is spaced apart from the second separation axis (L1) by a first distance (d1). The first ejection rods 331′ are spaced apart from each other along the second axis (X), and are located along the first separation axis (LX).

The second male mold units 32″ are separated by the first separation axis (LX) and a third separation axis (L2) that is perpendicular to the first separation axis (LX) and parallel to the second separation axis (L1) to form a 2×2 arrangement. The second male mold units 32″ located on two sides of the first separation axis (LX) are symmetrical mirror images of each other. The second male mold hole 323″ of each second male mold unit 32″ is located between the first separation axis (LX) and the second male mold surface 322″ of a respective second male mold unit 32″, and is spaced apart from the third separation axis (L2) by a distance same as the first distance (d1). The second ejection rods 331″ are spaced apart from each other along the second axis (X), and are located along the first separation axis (LX).

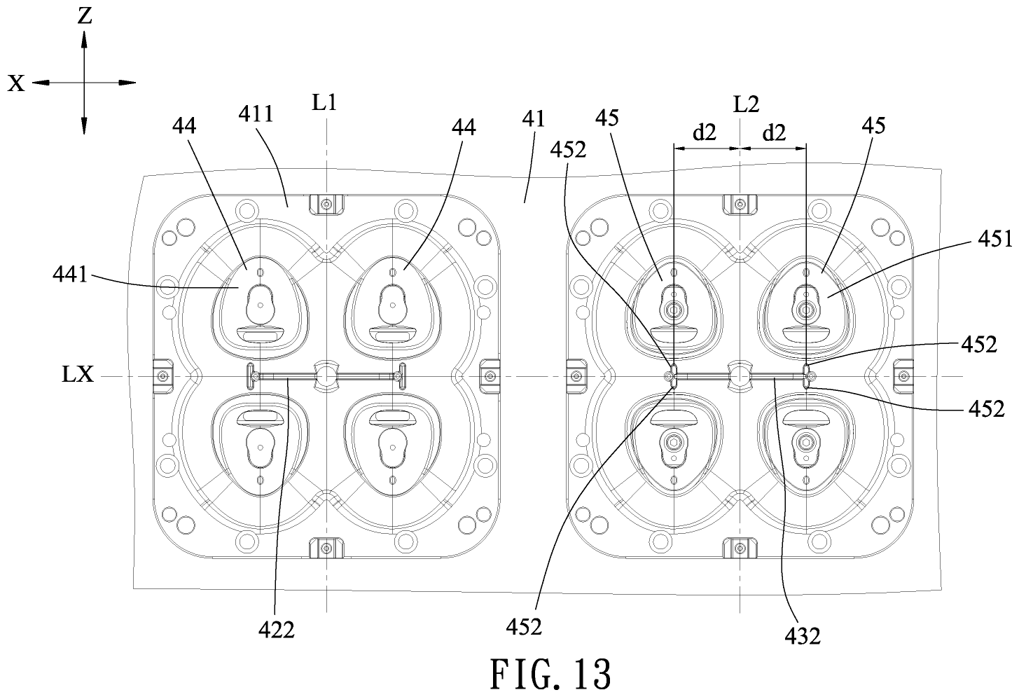

Referring to FIG. 13, in combination with FIGS. 10 and 11, the female mold 4 includes a female seat body 41, a first channel unit 42 formed in the female seat body 41 for injection of the first composition, a second channel unit 43 formed in the female seat body 41 and spaced apart from the first channel unit 42 along the second axis (X) for injection of the second composition, and four first female mold units 44 and four second female mold units 45 disposed on the female seat body 41.

The female seat body 41 has a second inner wall 411 and a second outer wall 412 opposite to each other along the first axis (Y). The first channel unit 42 has a first feeding channel 421 extending from the second outer wall 412 to the second inner wall 411, and a first runner 422 formed on the second inner wall 411 and extending generally along the second axis (X). The first runner 422 is transverse to and communicates with the first feeding channel 421, and corresponds in position to the first ejection rods 331′. The second channel unit 43 has a second feeding channel 431 extending from the second outer wall 412 to the second inner wall 411, and a second runner 432 formed on the second inner wall 411 and extending generally along the second axis (X). The second runner 432 is transverse to and communicates with the second feeding channel 431, and corresponds in position to the second ejection rods 331″.

The first female mold units 44 are spaced apart from the second female mold units 45 along the second axis (X). The first female mold units 44 are separated by the first separation axis (LX) and the second separation axis (L1) to form a 2×2 arrangement. The first female mold units 44 located on two sides of the first separation axis (LX) are symmetrical mirror images of each other. Each first female mold unit 44 has a first female mold surface 441 projecting from the second inner wall 411.

The second female mold units 45 are separated by the first separation axis (LX) and the third separation axis (L2) to form a 2×2 arrangement. The second female mold units 45 located on two sides of the first separation axis (LX) are symmetrical mirror images of each other. Each second female mold unit 45 has a second female mold surface 451 projecting from the second inner wall 411, and a female mold hole 452 formed in the second inner wall 411 and located between the first separation axis (LX) and the second female mold surface 451 of a respective second female mold unit 45. Specifically, the female mold holes 452 of the second female mold units 45 are arranged in sets of two respectively located on two opposite sides of the third separation axis (L2) and respectively communicating with two opposite ends of the second runner 432. Each female mold hole 452 is adjacent to the second female mold surface 451 of the respective second female mold unit 45, and is spaced apart from the third separation axis (L2) by a second distance (d2) that is less than the first distance (d1).

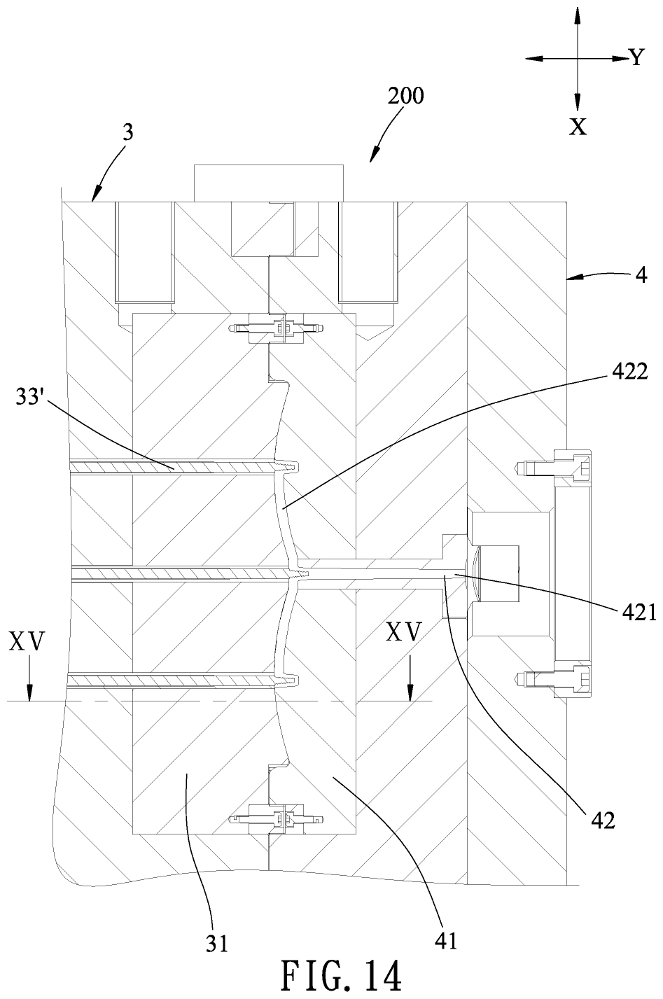

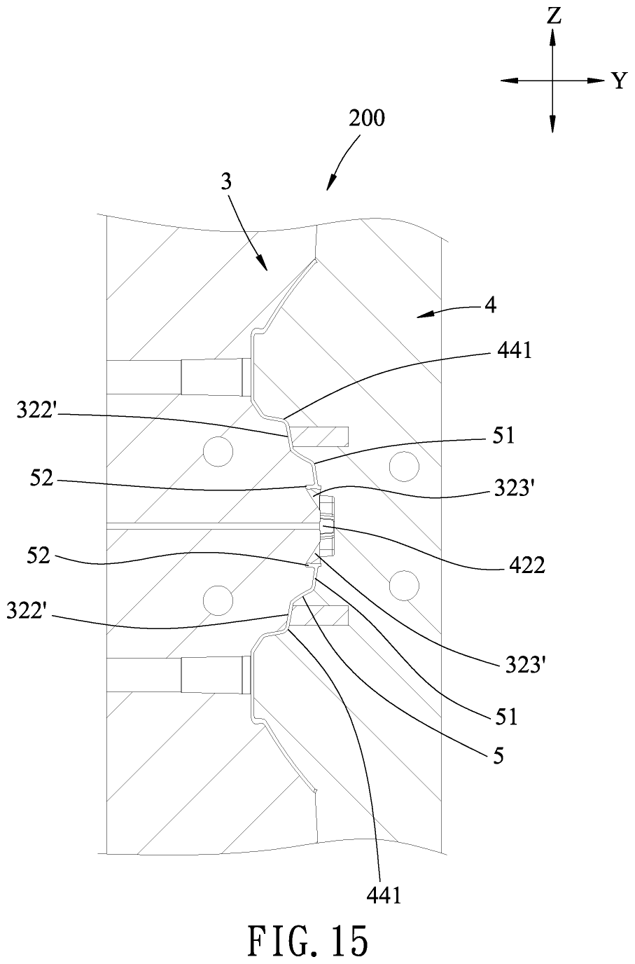

Referring to FIGS. 14 and 15, in combination with FIGS. 12 and 13, a process of making the respiratory mask 100 by cooperation of the male mold 3 and the female mold 4 of the mold 200 of this disclosure is described hereinbelow, and includes steps 1 to 7.

In step 1, with reference to FIGS. 12 to 15, the male mold 3 is moved toward the female mold 4 along the first axis (Y) to place the mold 200 in a first closed position. At this time, the first female mold surface 441 of each first female mold unit 44 extends into the first mold cavity 321′ of a respective first male mold unit 32′, and the first male mold surface 322′ of each first male mold unit 32′ and the first female mold surface 441 of the respective first female mold unit 44 cooperatively define a first mold chamber 5 which includes a main body region 51, and an extended region 52 extended from the main body region 51 toward the first male mold surface 322′. Furthermore, the first male mold hole 323′ of each first male mold unit 32′ communicates with the extended region 52 of the first mold chamber 5 defined by the first male mold surface 322′ of each first male mold unit 32′ and the first female mold surface 441 of the respective first female mold unit 44, and the first male mold holes 323′ of the first male mold units 32′ are arranged in sets of two respectively communicating with two opposite ends of the first runner 422.

In step 2, with reference to FIGS. 10 and 12 to 15, the first composition is injected through the first feeding channel 421, the first runner 422 and the first male mold holes 323′ of the first male mold units 32′ into the first mold chambers 5 respectively defined by the first male mold surfaces 322′ of the first male mold units 32′ and the first female mold surfaces 441 of the first female mold units 44, and is cooled to form four mask bodies 1 (see FIG. 11), and a first residue 7 (see FIG. 11) connected to the mask bodies 1. The shield portion 11 and the flange 12 of each mask body 1 are formed in the main body region 51 of each first mold chamber 5. The grip portion 15 of each mask body 1 is formed on the extended region 52 of each first mold chamber 5. The jaw section 113 of each mask body 1 is adjacent to a respective one of the first male mold holes 323′. The nose bridge section 114 of each mask body 1 is farther from the first separation axis (LX) than the jaw section 113. The first residue 7 is formed by the first composition that is cooled in the first feeding channel 421 and the first runner 422.

In step 3, the male mold 3 is moved away from the female mold 4 along the first axis (Y), and the first ejection rods 331′ are moved to extend out of the male seat body 31 and push the first residue 7 so as to detach the first residue 7 from the mask bodies 1 and falls naturally. At this time, the mask bodies 1 are respectively retained in the first mold cavities 321′. It should be noted that, for convenience of explanation, the mask bodies 1 are shown as floating in FIG. 11, but during the actual making process, the mask bodies 1 are respectively located in the first mold cavities 321′. Since each first male mold hole 323′ communicates with the extended region 52 of the respective first mold chamber 5, when the first residue 7 is separated from the mask bodies 1, gate scars (not shown) at junctions of the first residue 7 and the mask bodies 1 are formed on the grip portions 15 of the mask bodies 1. That is, during the injection molding process, the gate scars can all be formed on the grip portions 15, so as to prevent the gate scars from being formed on other portions of the mask bodies 1 to thereby reduce waste of excess residues.

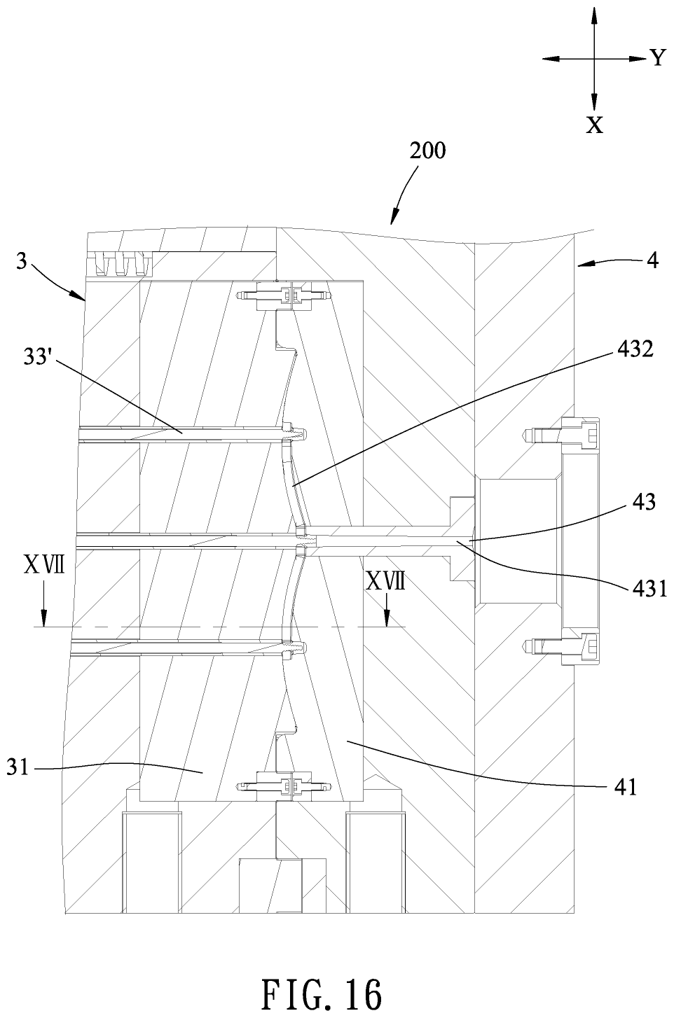

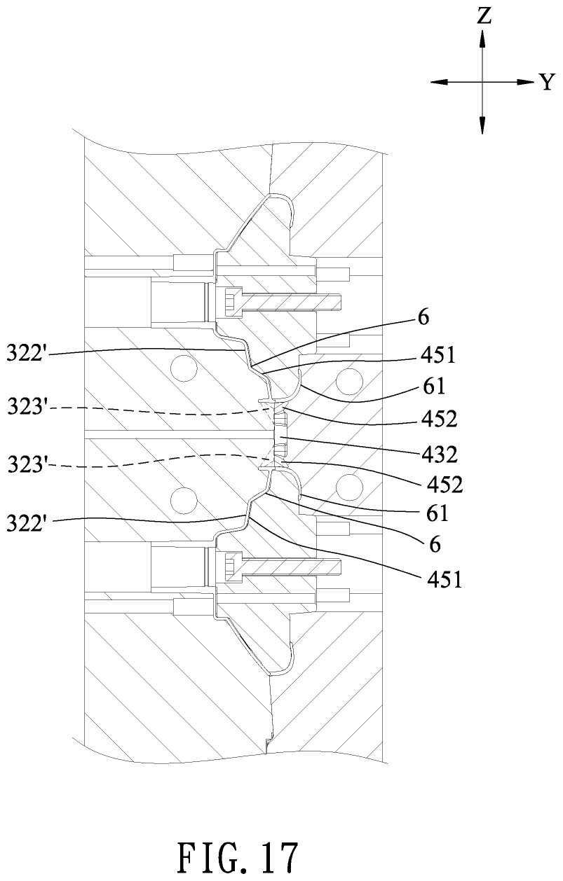

In step 4, with reference to FIGS. 11, 12, 16 and 17, the male mold 3 is rotated 180 degrees about the first axis (Y), after which it is again moved toward the female mold 4 along the first axis (Y) to place the mold 200 in a second closed position, and the second female mold surfaces 451 of the second female mold units 45 respectively extend into the first mold cavities 321′. Each first male mold surface 322′ and a respective second female mold surface 451 cooperatively define a product chamber 6 and a buffer chamber 61 communicating with the product chamber 6 and the female mold hole 452. Furthermore, the first female mold surfaces 441 respectively extend into the second mold cavities 321″ of the second male mold units 32″, and each first female mold surface 441 cooperates with the second male mold surface 322″ of the respective second male mold unit 32″ to define a second mold chamber (not shown) having a shape identical to that of the first mold chamber 5 shown in FIG. 15. Moreover, each first male mold hole 323′ and the female mold hole 452 of the respective second female mold unit 45 are adjacent to each other and are located proximate to a junction of the product chamber 6 and the buffer chamber 61. The product chamber 6 also has a shape identical to that of the first mold chamber 5.

In step 5, the second composition is injected through the second feeding channel 431, the second runner 432 and the female mold holes 452 of the second female mold units 45 into the buffer chambers 61 respectively defined by the first male mold surfaces 322′ and the second female mold surfaces 451, and is cooled to form four buffer members 2 respectively connected to the mask bodies 1, and a second residue 8 connected to the buffer members 2 (see FIG. 11). Each buffer member 2 and a respective one of the mask bodies 1 cooperatively form a respiratory mask 100. Moreover, the first composition is injected through the first feeding channel 421, the first runner 422, and the second male mold holes 323″ of the second male mold units 32″ into the second mold chambers, and is cooled to form another new four mask bodies 1 (see FIG. 11), and another first residue 7 (see FIG. 11) connected to the new mask bodies 1.

In step 6, the male mold 3 is moved away from the female mold 4 along the first axis (Y), four push rods (not shown) in the male mold 3 are moved to push the respiratory masks 100 out of the first mold cavities 321′, and the first ejection rods 331′ are moved to push the second residue 8 out of the male seat body 31, so that the respiratory masks 100 and the second residue 8 are separated from the male seat body 31. The second ejection rods 331″ are also moved to push the first residue 7 (see FIG. 11) that is connected to the newly formed four mask bodies 1 (see FIG. 11) so as to detach the first residue 7 from the mask bodies 1 and falls naturally.

In step 7, steps 4 to 6 are repeated to carry out the next round of making the respiratory masks 100.

In the mold 200 for making the respiratory mask 100 of this disclosure, since the second distance (d2) between each female mold hole 452 and the third separation axis (L2) is less than the first distance (d1) between each first male mold hole 323′ and the second separation axis (L1), when the male mold 3 is moved toward the female mold 4 to close the mold 200 in step 4, the second runner 432 will not communicate with the first male mold holes 323′, and the second composition that flows through the second runner 432 into the female mold holes 452 will not enter the first male mold holes 323′, thereby preventing the second composition from contaminating the mask bodies 1. Furthermore, since the structure of the mold 200 is a two-plate mold, it has the advantages of small size, simple structure, and low production cost. Moreover, since there is no hot runner structure in this disclosure, the temperatures of the mold 200 and the product will not be affected by the fluid temperature in the runners, so that the molding quality of the product is stable.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A respiratory mask, comprising:
a mask body injection molded from a first composition and including a shield portion that surrounds a central axis and that has an air inlet, a flange extending outwardly from an outer periphery of said shield portion, and a grip portion disposed on said flange and cooperating with said flange and said shield portion to define a receiving groove, said shield portion defining a facial space communicating with said air inlet, said flange having an inner surface proximate to said facial space, and an outer surface opposite to said inner surface, said grip portion protruding from said outer surface of said flange in a direction parallel to said central axis; and
a buffer member that is substantially ring-shaped, that is injection molded from a second composition, that is connected to and surrounds said mask body and that has a hardness smaller than that of said mask body.

2. The respiratory mask as claimed in claim 1, wherein said shield portion includes an intermediate section, an air tube that is connected to said intermediate section, that defines said central axis and that has said air inlet, a jaw section located on one side of said intermediate section and connected to said flange, and a nose bridge section located on another side of said intermediate section and opposite to said jaw section along a transverse direction transverse to said central axis, said grip portion cooperating with said flange and said jaw section to define said receiving groove.

3. The respiratory mask as claimed in claim 2, wherein said grip portion has an elongated plate shape extending in a direction transverse to said transverse direction, said grip portion having a first side surface adjacent to said receiving groove and facing said jaw section, a second side surface opposite said first side surface, and a connecting surface connected between ends of said first side surface and said second side surfaces that are distal from said flange.

4. The respiratory mask as claimed in claim 2, wherein said grip portion has a plurality of protrusions protruding from said outer surface of said flange and spacedly arranged in a direction transverse to said transverse direction.

5. The respiratory mask as claimed in claim 2, wherein said grip portion has an elongated section connected to said outer surface of said flange and extending in a direction transverse to said transverse direction, and a groove recessed in and extending along a length of said elongated section.

6. The respiratory mask as claimed in claim 2, wherein said grip portion has an elongated section connected to said outer surface of said flange and extending in a direction transverse to said transverse direction, and a plurality of protruding sections extending from said elongated section toward said jaw section and spaced apart from each other along a length of said elongated section.

7. The respiratory mask as claimed in claim 1, wherein said shield portion includes an intermediate section, a jaw section located at one side of said intermediate section, and a nose bridge section located at another side of said intermediate section and opposite to said jaw section along said transverse direction, said flange extending along an outer periphery of said shield portion from one side of said intermediate section through said jaw section to the other side of said intermediate section that is opposite to said one side along a direction transverse to said transverse direction, said grip portion having four protrusions, one of said four protrusions being located on said flange adjacent to said jaw section, the other one of said four protrusions being located on said nose bridge section, the remaining two of said four protrusions being located on said flange and respectively adjacent to two sides of said intermediate section that are opposite along a direction transverse to said central axis.

8. A mold for making at least one two-color molded anti-slip grip respiratory mask, said at least one two-color molded anti-slip grip respiratory mask including a mask body configured to be injection molded from a first composition, and a buffer member configured to be injection molded from a second composition, said mask body including a shield portion, a flange connected to said shield portion, and a grip portion protruding from said flange, said buffer member being connected to and surrounding said mask body and having a hardness smaller than that of said mask body, said mold comprising:
a male mold including a male seat body having a first inner wall, and at least one first male mold unit disposed on said male seat body, said at least one first male mold unit having a first mold cavity extending inwardly from said first inner wall and defined by a first male mold surface, and a first male mold hole formed in said first inner wall and adjacent to and communicating with said first mold cavity; and
a female mold including a female seat body having a second inner wall, a first channel unit formed in said female seat body for injection of the first composition, a second channel unit formed in said female seat body for injection of the second composition and spaced apart from said first channel unit, at least one first female mold unit disposed on said female seat body, and at least one second female mold unit disposed on said female seat body and spaced apart from said at least one first female mold unit, said at least one first female mold unit having a first female mold surface projecting from said second inner wall, said at least one second female mold unit having a second female mold surface projecting in from said second inner wall, and a female mold hole formed in said second inner wall adjacent to said second female mold surface and communicating with said second channel unit, when said male mold is moved toward said female mold to place said mold in a first closed position, said first male mold surface and said first female mold surface cooperatively define a first mold chamber communicating with said first male mold hole and said first channel unit for forming said mask body, said first mold chamber including a main body region for forming said shield portion, and an extended region extended from said main body region toward said first male mold surface for forming said grip portion, said extended region being adjacent to and communicating with said first male mold hole; and
when said male mold is moved toward said female mold to place said mold in a second closed position, said first male mold surface and said second female mold surface cooperatively define a product chamber and a buffer chamber communicating with said product chamber and said female mold hole for forming said buffer member.

9. The mold as claimed in claim 8, wherein said male mold is moved toward said female mold along a first axis to close said mold, and when said mold is in said second closed position, said first male mold hole and said second male mold hole are adjacent to each other and are located proximate to a junction of said product chamber and said buffer chamber.

10. The mold as claimed in claim 8, wherein:
said male mold is moved toward said female mold along a first axis to close said mold;
said male mold includes four said first male mold units which are separated by a first separation axis and a second separation axis to form a 2×2 arrangement, said first separation axis extending parallel to a second axis that is perpendicular to said first axis, said second separation axis extending parallel to a third axis that is perpendicular to said first and second axes; said first male mold hole of each of said first male mold units being located between said first separation axis and said first male mold surfaces of a respective one of said first male mold units, and being spaced apart from said second separation axis by a first distance, and
said female mold includes four said first female mold units and four said second female mold units, said first female mold units being spaced apart from said second female mold units along said second axis, said first female mold units being separated by said first separation axis and said second separation axis to form a 2×2 arrangement, said second female mold units being separated by said first separation axis and a third separation axis that is parallel to said second separation axis to form a 2×2 arrangement, said female mold hole of each of said second female mold units being located between said first separation axis and said second female mold surface of a respective one of said second female mold units, and communicating with said second channel unit, said female mold hole of each of said second female mold units being adjacent to said second female mold surface of said respective one of said second female mold units, and being spaced apart from said third separation axis by a second distance that is less than said first distance.
